(19) United States
(12) Patent Application Publication
(10) Pub. No.: US 2020/0405560 A1
(43) Pub. Date: Dec. 31, 2020

(54) MEDICAL BASIN WITH APPENDAGE NOTCH

(71) Applicant: Tobias Cross, Urbandale, IA (US)

(72) Inventor: Tobias Cross, Urbandale, IA (US)

(21) Appl. No.: 16/456,288

(22) Filed: Jun. 28, 2019

(51) Int. Cl. A61G 9/02; A61B 50/30

(52) U.S. Cl. CPC A61G 9/02; A61G 7/047; A61B 50/30

(57) ABSTRACT

A portable medical basin contains blood, other liquids, and other supplies and materials used in treating a wound to a patient's hand, arm, foot, or leg. The human or animal appendage or limb is positioned in a molded notch in one side wall of the basin or may be extended through opposing notches in opposite side walls of the basin, such that the wound is positioned over the basin. Then, the blood from the wound and liquids used in cleaning and treating the wound are captured in the basin for proper disposal. The notch includes a widened lip upon which the leg or arm rests. The lip minimizes discomfort, scratching and cutting of the arm or leg during the medical procedure.

### FIELD OF THE INVENTION

The invention is directed to a medical device for collecting fluids, bandages, cleaning supplies, and other materials during a medical procedure in a safe and comfortable manner. In particular, the device is a tub or basin having a molded notch with a widened edge, with a patient's limb being received in the notch while the appendage is inspected and/or a procedure is performed on the arm, hand, leg, or foot residing over the tub or basin.

### BACKGROUND OF THE INVENTION

Medical personnel, such as doctors, nurses, physician assistants, and others who work directly with patients, are sometimes faced with injuries that need immediate attention. For example, a cut or wound on a person's arm, leg, hand, or foot which is bleeding needs prompt attention to stop the bleeding, and to inspect, clean and dress the wound. Stitches may be required to close the wound. Blood dripping from the wound needs to be contained. Also, water, saline, or other liquids used to clean the wound should be collected for proper disposal.

Typically, a container, such as a basin, is often used in a position beneath the arm or leg to catch the blood and other fluids or liquids used by the medical personnel treating the wound. Depending upon the location of the wound, the arm or leg may be difficult to retain in place over the basin or the weight of the limb makes the basin unstable and subject to tipping. It has been known to use a pair of scissors to cut a V-shaped notch in a plastic basin or tub, such that the patient's arm or leg can extend through the notch so that the wound resides over the basin. However, cutting the notch leaves a sharp, narrow edge which can further injure the patient and provide an even more uncomfortable situation while the wound is being treated.

Therefore, there is a need in the medical industry for an improved catch basin for fluids and other materials, and having a notch or recess with a molded, smooth edge to minimize or eliminate discomfort to a human or animal patient whose arm or leg rests within the notch while a wound or injury is being treated by the medical personnel, and which is safe and stable in use.

Accordingly, a primary objective of the present invention is the provision of an improved medical basin which comfortably receives an appendage of a patient by treating a wound or injury to the appendage.

Another objective of the present invention is the provision of a medical basin having a molded notch with an edge having an increased diameter to minimize discomfort to a patient's arm or leg residing in the notch, and to reduce or eliminate added injury or discomfort from a sharp edge.

A further objective of the present invention is the provision of a medical basin having a molded notch on one end, or a pair of molded notches on opposite ends, through which a patient's arm or leg extends.

Still another objective of the present invention is the provision of a method of containing fluids and waste materials in a medical procedure on a human or animal limb, wherein the limb is positioned into a portable basin having sidewalls with at least one molded U-shaped notch with a portion of the appendage resting in the notch, such that the wound or injury is over the basin to capture blood from the wound, and to capture fluids used in treating the wound and other waste products.

Yet another objective of the present invention is the provision of a portable medical basin sized to receive a human or animal arm or leg, with the edge of the notch having a thickness greater than the thickness of the sidewall.

A further objective of the present invention is the provision of a medical basin or tub which can be sterilized for repeated uses or disposed after a single use.

Still another objective of the present invention is the provision of a portable tub for medical treatments and procedures which can be easily stacked for storage and shipping.

Another objective of the present invention is the provision of an improved, lightweight medical basin tub which is economical to manufacture, stable in use and is safe and comfortable to the patient.

These and other objectives will become apparent from the following description of the invention.

### SUMMARY OF THE INVENTION

A portable, lightweight, medical basin includes a molded body having a flat bottom wall and upstanding sidewalls, with an open top. A U-shaped notch is molded into one of the sidewalls and has a depth sufficient to receive an arm or leg of a patient. The edge of the notch includes a lip or flange which extends substantially perpendicular to the sidewalls with the notch, and which provides a smooth, widened edge upon which the arm or leg can rest, without risk of being cut, scratched, or otherwise irritated. A second U-shaped notch may be provided in the opposite sidewall, such that the appendage can extend across the tub, with the wound or injury being positioned above the bottom wall. The bottom of the notch is spaced above the bottom wall, such that blood and other fluids or liquids can be contained within the basin.

The notched basin provides a method of containing fluids during a medical procedure on the arm or leg, by positioning the arm or leg into the notch, such that the wound or injury resides between the sidewalls of the basin so as to capture the fluids draining from the wound or injury, and other cleaning or treating liquids applied to the arm or leg during treatment of the wound or injury.

The basin can be used by doctors, nurses, physician assistants, veterinarians, and other medical staff in offices clinics, hospitals, and by others at home, for treating injuries to humans and animals.

### BRIEF DESCRIPTION OF THE DRAWINGS

FIG. 1 is a perspective view of a first embodiment of a medical basin according to the present invention, with a U-shaped notch molded in one sidewall of the basin.

FIG. 2 is an end elevation view of the basin, showing the molded notch.

FIG. 3 is a sectional view taken along lines 3-3 of FIG. 2.

FIG. 3A is a sectional view showing an alternative embodiment of the notch having a radius.

FIG. 4 is a perspective view of a second embodiment of the medical basin, with notches formed at opposite sides of the basin.

FIG. 5 is a perspective view of a prior art basin having a V shaped notch cut out of one wall using scissors or a knife.

### DETAILED DESCRIPTION OF THE INVENTION

A medical basin or tub is generally designated in the drawings by the reference numeral 10. The basin 10 is preferable made of medical grade plastic, or alternatively, stainless steel or other lightweight material suitable for medical environments. The basin 10 is formed as one-piece so as to contain fluid or liquids, without the risk of leakage. The basin 10 can also hold other materials, such as bandages, sutures, cleaning supplies, and small medical equipment used on a wound. The basin 10 includes a bottom wall 12 and side walls 14, 16, 18, and 20. The top of the basin is open. The upper edge or perimeter of the basin 10 includes a molded or formed lip 22 extending substantially perpendicular to the upright side walls 14, 16, 18, and 20. The sidewalls 14, 16, 18, 20 may be slightly tapered from top to bottom so that multiple basins 10 can be stacked for storage or transport.

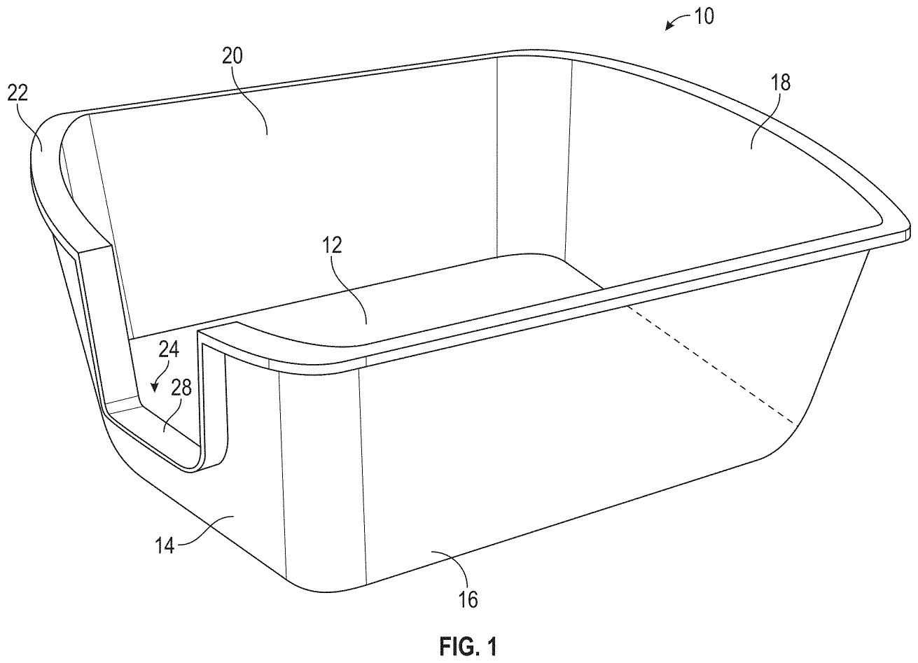

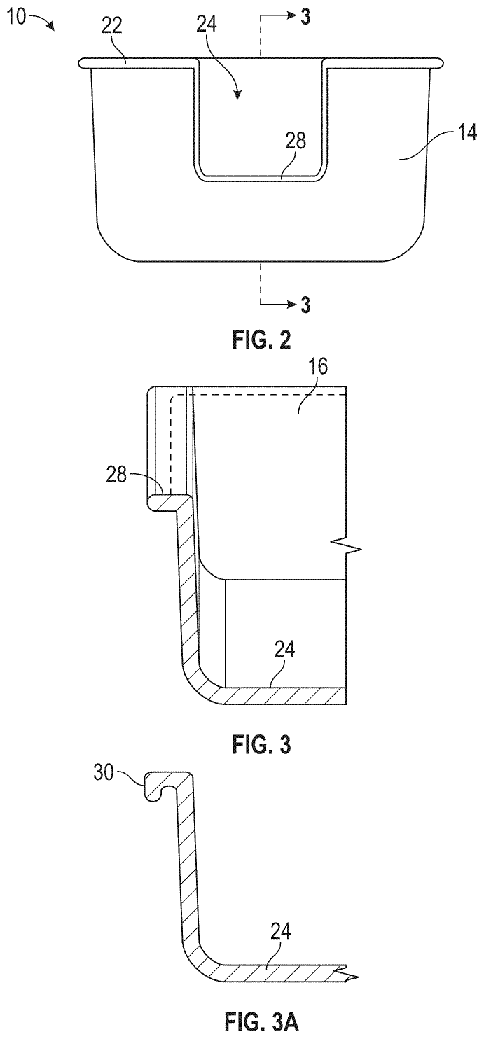

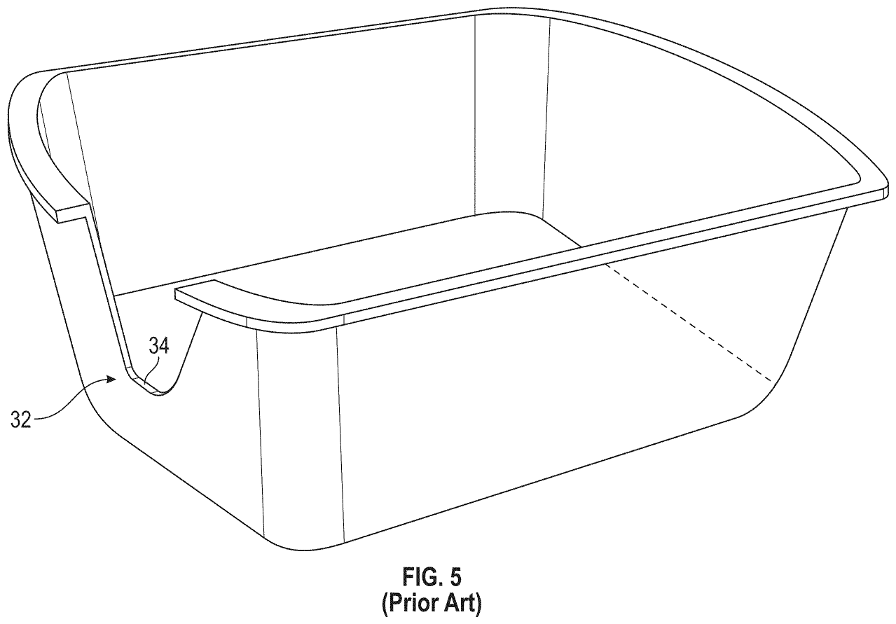

In the first embodiment, shown in FIGS. 1 and 2, a U-shaped notch or recess is molded or otherwise formed in the side wall 14, though it is understood that the notch 24 can be formed in any of the side walls. In an alternative embodiment, shown in FIG. 4, a second notch 26 is formed in the sidewall 18, opposite the first notch 24. The opposing notches 24, 26 may be formed with the same size, or with different sizes or depths.

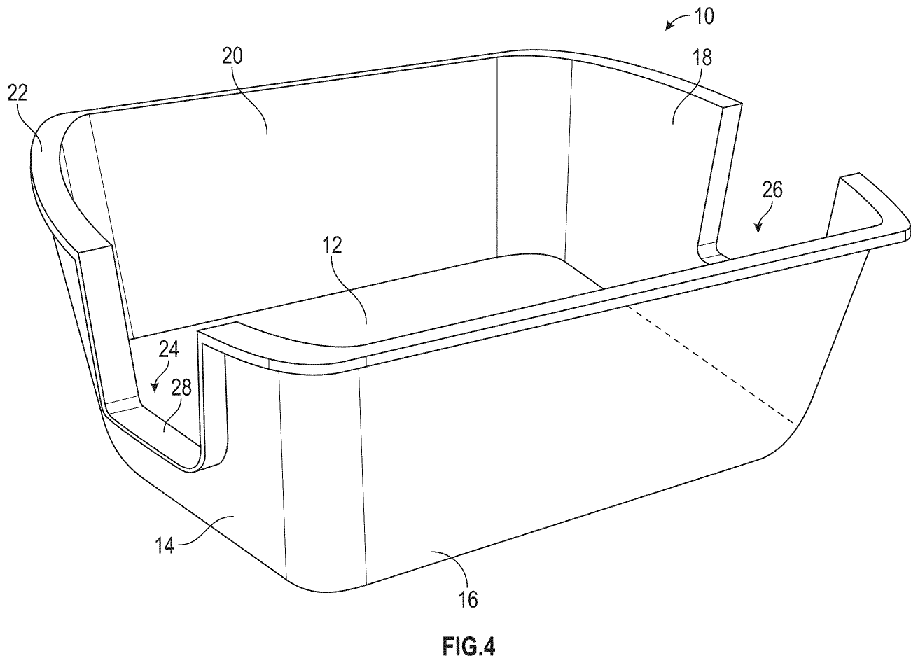

The notches 24, 26 are molded or formed with a lip 28 extending along the opposite sides and the bottom of the notch. The lip 28 preferably is a continuation of the lip 22 and extends substantially perpendicular to the side wall in which the notch 24, 26 is formed. The lip 28 provides a smooth notch surface having an increased thickness or width greater than the thickness of the side wall 14, 18. The lip 28 defines an L-shaped edge for the notches 24, 26, as shown in FIG. 3. In an alternative embodiment, shown in FIG. 3A, the notches 24, 26 may have an inverted U-shaped lip 30 with a downwardly turned end, and/or may have a curved upper radius.

The basin 10 can be used by anyone treating an injury on a human or animal arm, hand, leg, or foot. The basin may be made in various sizes to accommodate different patients or different appendages/limbs. The basin to can be made of any suitable material, such as re-usable stainless steel, or disposable plastic or composite.

In use, when a patient has a wound or injury on their hand, arm, foot, or leg, the arm or leg can be positioned in the notch 24, or may extend through both notches 24, 26, such that the wound or injury resides above the bottom 12 and within the side walls 14, 16, 18, 20 of the basin 10. Any blood dripping from the wound will be captured in the basin 10. Also, fluids or liquids used by medical personnel to clean or treat the wound, and other materials and equipment, will also be retained in the basin 10. After the medical procedure is completed, the patient's arm or leg can be removed from the notch 24 or notches 24, 26, such that the contents of the basin can be disposed of safely. A weight may be attached or added to the basin opposite the notch 24 to stabilize the basin and prevent tipping.

Preferably, the basin 10 is sterilized before use with the patient. After use, the basin 10 may be re-sterilized for future use or may be discarded after a single use, depending on the basin construction material.

The smooth or rounded edge provided by the lip 28 or 30 prevents cutting, scratching, or irritation to the patient's arm or leg while the appendage rests upon the lip 28 or 30. In comparison, in the prior art, shown in FIG. 5, a notch 32 was created using scissors or a knife, which left a rough or sharp edge 34. The thin edge 34, which had a thickness corresponding to the thickness of the sidewall which was cut, could easily cut or scratch patient's skin, or otherwise irritate the arm or leg while the patient's wound was being treated. Such additional injury or discomfort to the patient is highly undesirable, and is completely eliminated by the molded notch 24, or pair of notches 24, 26, with the relatively wide lip 28, 30.

The invention has been shown and described above with the preferred embodiments, and it is understood that many modifications, substitutions, and additions may be made which are within the intended spirit and scope of the invention. From the foregoing, it can be seen that the present invention accomplishes at least all of its stated objectives.

What is claimed is:

1. A portable medical basin, comprising:
a molded body having a flat bottom wall and upstanding side walls, and an open top;
a first U-shaped notch molded in one of the side walls, and having a depth sufficient to receive a human limb.

2. The medical basin of claim 1 wherein the notch has a lip extending substantially perpendicular to the one side wall.

3. The medical basin of claim 1 wherein the notch has a rounded edge.

4. The medical basin of claim 1 wherein the notch is spaced above the bottom wall.

5. The medical basin of claim 1 wherein a second notch is molded in another of the sidewalls opposite the first notch.

6. The medical basin of claim 5 wherein both notches have a perimeter lip.

7. The medical basin of claim 5 wherein both notches include a rounded edge.

8. The medical basin of claim 5 wherein the notches have identical shapes and sizes.

9. The medical basin of claim 5 wherein the notches have different sizes.

10. The medical basin of claim 1 wherein the sidewalls have a height sufficient to retain fluids draining from or applied to the limb.

11. A method of containing fluids during a medical procedure on a human body appendage, comprising:
positioning the appendage into a portable basin having sidewalls with at least one molded U-shaped notch, and a portion of the appendage residing in the notch;
resting the appendage on a widened lip on the notch greater than a thickness of the sidewall in which the notch exits; and
capturing fluids used in treating the appendage in the basin for later disposal.

12. The method of claim 11 further comprising extending the appendage through a second notch in an opposite sidewall of the basin.

13. The method of claim 12 wherein positioning a wound on the appendage over the basin contains blood from the wound, and cleaning liquids and supplies used in treating the wound.

14. The method of claim 12 wherein the notch has a width greater than a width of the appendage so that side edges of the notch are spaced from the appendage.

15. A portable medical basin for containing liquids, waste materials, and medical tools and supplies, comprising:
a body with sidewalls;
a notch formed in one of the sidewalls and sized to receive a human leg or arm;
the notch being molded into the one sidewall with a horizontal edge having a thickness greater than a thickness of the sidewall.

16. The portable medical basin of claim 15 wherein the notch edge is a lip extending perpendicular to the one sidewall.

17. The portable medical basin of claim 16 wherein the body has a flat bottom.

18. The portable medical basin of claim 17 wherein the body is sterilized.

19. The portable medical basin of claim 18 wherein the body is plastic.

20. The portable medical basin of claim 18 wherein the sidewalls are tapered for stacking additional basins.
